(19) United States
(12) Patent Application Publication
(10) Pub. No.: US 2025/0121847 A1
(43) Pub. Date: Apr. 17, 2025

(54) Assistance System and Assistance Method for a Vehicle

(71) Applicant: Bayerische Motoren Werke Aktiengesellschaft, Muenchen (DE)

(72) Inventor: Etienne ILIFFE-MOON, Menlo Park, CA (US)

(21) Appl. No.: 18/692,461

(22) PCT Filed: Mar. 15, 2023
(86) PCT No.: PCT/EP2023/056621

§ 371 Date: Mar. 15, 2024

(30) Foreign Application Priority Data
Mar. 28, 2022 (DE) 10 2022 107 293.2

(51) Int. Cl. B60W 50/16; B60W 40/08; B60W 50/14; G06F 3/16; G06V 20/59; G06V 40/16; G06V 40/20

(52) U.S. Cl. CPC B60W 50/16; B60W 40/08; G06F 3/165; G06V 20/597; G06V 40/174; G06V 40/20; B60W 2040/089; B60W 2050/143; B60W 2050/146; B60W 2420/403; B60W 2420/54; B60W 2540/21

(57) ABSTRACT

An assistance system for a vehicle is provided that includes an output module, which is configured for music output in a vehicle interior, on the basis of musical acoustic data, and an interior sensor system that is configured to optically and/or acoustically detect at least one vehicle occupant and to provide corresponding detection data. The assistance system is configured to determine a reaction of the at least one vehicle occupant to the music output on the basis of the detection data; and activate at least one vehicle function on the basis of the reaction of the at least one vehicle occupant to the music output, in order to provide the at least one vehicle occupant with feedback on the reaction to the music output.

### BACKGROUND AND SUMMARY

The present disclosure relates to an assistance system for a vehicle, a vehicle having such an assistance system, an assistance method and a storage medium for performing the assistance method. The present disclosure relates in particular to an intelligent interaction of the assistance system with vehicle occupants with regard to played-back media content, for example, as part of a sing function or karaoke function of the assistance system.

The development of driving assistance functions, for example, for (partially) autonomous driving, e.g., according to SAE level 3, 4 or 5, is becoming ever more important. Active drivers are often required only intermittently or not at all in such vehicles, and so the question increasingly arises as to how vehicle occupants can be kept occupied during (partially) autonomous driving. One possible approach involves providing the vehicle occupants with infotainment offers to keep them busy during the (partially) autonomous journey. However, conventional infotainment offers such as films or video games have limited options for keeping the vehicle occupants busy.

It is an object of the present disclosure to specify an assistance system for a vehicle, a vehicle having such an assistance system, an assistance method and a storage medium for performing the assistance method that facilitate an intelligent interaction of the assistance system with vehicle occupants with regard to played-back media content. In particular, it is an object of the present disclosure to provide a sing function, e.g., a karaoke function, in a vehicle.

This object is achieved by the subject matter of the independent claims. Advantageous configurations are specified in the dependent claims.

According to one independent aspect of the present disclosure, an assistance system for a vehicle, in particular a motor vehicle, is specified. The assistance system comprises an output module designed for music output, or music reproduction, on the basis of musical acoustic data in a vehicle interior; and an interior sensor system designed to optically and/or acoustically detect at least one vehicle occupant and to provide corresponding detection data. The assistance system is further designed to take the detection data as a basis for determining a reaction, or behavior, of the at least one vehicle occupant in response to the music output; and to take the reaction, or behavior, of the at least one vehicle occupant in response to the music output as a basis for controlling at least one vehicle function in order to provide the at least one vehicle occupant with feedback relating to the reaction in response to the music output.

According to the invention, a reaction, or behavior, of a vehicle occupant in response to played-back media content is detected and analyzed, e.g., as part of a sing function or karaoke function. The analysis is taken as a basis for providing the vehicle occupant with qualified feedback in relation thereto. By way of example, a performance of a single vehicle occupant or of multiple vehicle occupants can be assessed (at the same time or in succession) when singing along to music by assessing the behavior (expression, movement, dynamics, etc.) of various facial and/or body features and the synchronization with the song text and/or the music. This can be used to facilitate an intelligent interaction of the assistance system with the vehicle occupant with regard to played-back media content, e.g., as part of a sing function or karaoke function.

The process according to the invention, for example, the sing function or karaoke function of the assistance system, can be used by any suitable number of persons inside and outside the vehicle. In particular, a single vehicle occupant may play alone or, e.g., compete against the assistance system (e.g., an intelligent personal assistant (IPA), a digital or virtual person, an avatar of a famous person, etc.). In another example, vehicle occupants may compete against one another (e.g., individually or in teams) and/or against the IPA. In yet another example, at least one vehicle occupant may compete against at least one person outside the vehicle. The present disclosure is not limited thereto, however, and other lineups of vehicle occupants and/or IPA and/or external persons are conceivable for shared use of the assistance system, in particular the sing function or karaoke function.

Preferably, the music output, or playback of the music, is provided by way of at least one loudspeaker in the interior of the vehicle. In some embodiments, the output module may comprise the at least one loudspeaker. In other embodiments, the output module and the at least one loudspeaker may be separate units that are communicatively connected, the output module being able to output control signals to the at least one loudspeaker to play back the music.

Preferably, the assistance system is designed for zone audio, where different music and/or different texts can be provided for different users in different vehicle seats. Additionally or alternatively, the assistance system may be designed for spatial audio (e.g., Apple Spatial Audio, Dolby ATMOS, etc.), with, e.g., spatial audio components being filtered and output according to the singing person(s) (e.g., lead vs. backing vocals, multiple persons in a duet, etc.).

Preferably, the output module is further designed to visually output a linguistic part of the music output on the basis of lyrics data. In some embodiments, song texts can be visually displayed, for example, as writing, but the present disclosure is not limited thereto. The song texts may be optional and/or may not be made available to the vehicle occupant by the system or may be made available only under certain conditions (e.g., for advanced singers, depending on the selection of the vehicle occupant, depending on the mode of the assistance system, etc.; e.g., the assistance system may be designed to omit specific texts or lines of text, with the singer needing to remember these texts and sing them correctly).

In addition to or in combination with the display of the song text, some embodiments may involve one or more of the following being displayed: (i) a timing of the singing, (ii) what words/notes should be sung at a specific time, (iii) pauses between the texts (what should not be sung), (iv) a position of the singer in the song/text, (v) a cadence or rhythm of the song/text, (vi) a graphical aid (e.g., graphics, moving graphics, film excerpts, animations, etc.) that helps the user to be in sync with the song/text or the artist singing the song (e.g., conveyed by viewing a music video), (vii) a music video (in addition to the sound) and (viii) combinations of these.

The visual output of the song texts is preferably provided by means of a display apparatus. In some embodiments, the output module may comprise the display apparatus. In other embodiments, the output module and the display apparatus may be separate units that are communicatively connected, the output module being able to output control signals to the display apparatus to display the song texts.

Preferably, the display apparatus is a display of an infotainment system. Typically, the display is installed in or on the dashboard of the vehicle. The display may be a head unit, for example. In some embodiments, the display is an LCD display, a plasma display or an OLED display.

The present disclosure is not restricted to the examples mentioned and the output of the song texts may additionally or alternatively be provided using smart devices, e.g., using an app that can be shared with the vehicle, or a cloud-based system that operates in sync with devices and/or with the vehicle.

The musical acoustic data comprise data suitable for facilitating the music output, or music reproduction. The musical acoustic data may optionally or alternatively comprise video content or both audio and video content in a film format (e.g., music video, streaming music audio and/or video, etc.).

Similarly, the lyrics data comprise data suitable for visually reproducing song texts, e.g., as writing on a display. In addition, it is conceivable for an intelligent system (e.g., with IPA capability) to be able to detect the song text by “listening to” or analyzing the song (in advance and/or in real time and/or by way of retrieval from the past).

The assistance system can be implemented in at least two different ways: in a first implementation, the music and song texts are integrated in the assistance system. In a second implementation, the assistance system operates in parallel with the music and the song texts provided by another system (e.g., an infotainment player, a third-party app running in the vehicle, CarPlay, Android Auto, a mobile terminal, etc.).

With regard to the first implementation, the assistance system may comprise a storage module that stores, in particular permanently stores (as opposed to temporary buffer-storage in the case of streaming), the musical acoustic data and optionally the lyrics data.

In some embodiments, the storage module may be integrated in the vehicle or an external unit connected to the vehicle. By way of example, the assistance system may further comprise an interface module designed to receive the musical acoustic data and/or the lyrics data from the external unit. The interface module can communicate with the external unit by means of a wireless connection (e.g., Bluetooth, WLAN, NFC, etc.), for example.

Preferably, the external unit is a mobile terminal, e.g., of a vehicle occupant. The term mobile terminal covers in particular smartphones but also other mobile phones or mobiles, personal digital assistants (PDAs), tablet PCs and any current and future electronic devices equipped with a technology for loading and executing apps.

The assistance system with integrated music and song texts of the first implementation can work for example as an app inside the vehicle, by means of a projected mode (e.g., CarPlay or Android Auto) or from a mobile terminal.

With regard to the second implementation, the assistance system may be designed to receive the musical acoustic data and/or the lyrics data from an external unit and/or a streaming service. In other words, the music and/or the song texts are not integrated but rather are provided from outside as a stream. The assistance system thus operates in parallel with an existing music player and uses the music player (e.g., Apple Music and Spotify supply song texts while the song is being played back). This approach of operating in parallel with the source of the music/texts can simplify the assistance system and for example is not dependent on the provision of music and texts, the management of content or license issues.

Preferably, the interior sensor system comprises at least one interior camera and/or at least one interior microphone. The at least one interior camera can supply video data for analyzing reactions or the behavior of the vehicle occupant(s) (e.g., the singing performance). Similarly, the at least one interior microphone can supply audio data for analyzing the singing performance (further).

Preferably, the reaction of the at least one vehicle occupant in response to the music output comprises a physical behavior of the at least one vehicle occupant, for example a body expression, a body movement, a facial expression and/or a lip movement. By way of example, the assistance system can implement a facial recognition function in order to use the video data from the at least one interior camera to recognize facial characteristics, lip movements, etc., in time with the text and the music. In some embodiments, the assistance system can assess a facial expression of the vehicle occupant, for example, by virtue of the assistance system comparing the expression of the vehicle occupant with an original expression of the artist (which may require the assistance system to be trained for specific songs or a series of examples).

Additionally or alternatively, the reaction of the at least one vehicle occupant in response to the music output comprises an acoustic performance of the at least one vehicle occupant with regard to the music output, in particular a volume, a rhythm and/or a register for singing along.

Additionally or alternatively, the reaction of the at least one vehicle occupant in response to the music output comprises a synchronization between a singing along of the at least one vehicle occupant and the music output and/or the linguistic part of the music output. By way of example, the assistance system can analyze a degree of synchronization of the facial characteristics of one or more vehicle occupants (e.g., multiple vehicle occupants at the same time) with the text and/or music. The assistance system can implement lip reading (e.g., supported by AI/ML in order to train the assistance system to lip-read singing). In some embodiments, the degree of synchronization may comprise parameters such as timing and/or movement of the facial and/or body features (e.g., whether the mouth and lips move far enough apart for the given dynamics and volume of the music).

Additionally or alternatively, the reaction of the at least one vehicle occupant in response to the music output comprises an emotional state of the at least one vehicle occupant. The emotional state can provide an indication of a subjective perceived performance of the at least one vehicle occupant, for example.

Preferably, the assistance system further comprises an artificial intelligence (AI) module designed to take the detection data from the interior sensor system as a basis for determining the reaction, or behavior, of the at least one vehicle occupant in response to the music output.

Preferably, the artificial intelligence module comprises a trained algorithm. The algorithm can be trained by means of machine learning (ML) on the basis of training data, for example. The larger the training dataset, the better a performance of the assistance system becomes.

In some embodiments, the algorithm of the artificial intelligence module can be trained on the basis of video data and/or audio data from single or multiple persons singing to different songs.

Additionally or alternatively, the algorithm can be trained on the basis of musical acoustic data and/or lyrics data corresponding to the music output (i.e., the same songs).

Additionally or alternatively, the algorithm can be trained on the basis of musical acoustic data and/or lyrics data similar to the music output (i.e., similar songs).

Additionally or alternatively, the algorithm can be trained on the basis of historical data with regard to earlier music outputs and reactions of vehicle occupants in response thereto. In particular, the assistance system may comprise an integrated training function, with the result that the assistance system learns from the behavior of the actual users to a certain degree during the life of the assistance system.

Preferably, the assistance system is designed to output the feedback to the at least one vehicle occupant audibly and/or visually and/or haptically. In particular, the at least one vehicle function can be controlled on the basis of the reaction of the at least one vehicle occupant in response to the music in order to output audible and/or visual and/or haptic feedback.

The audible feedback can be provided, for example, by way of music, sounds, tones, sound effects, voice (e.g., by way of an intelligent personal assistant, IPA) or combinations of these. The present disclosure is not limited thereto, however, and other audible means can be used in order to output the feedback to the at least one vehicle occupant.

The visual feedback can be provided for example by way of a display apparatus, for example a head-up display (HUD), a projection HUD (PHUD) and/or a center information display (CID). The present disclosure is not limited thereto, however, and other display apparatuses, e.g. other graphical user interfaces (GUIs) and/or digital projectors, can be used in order to output the feedback to the at least one vehicle occupant.

Additionally or alternatively, the visual feedback can be provided by way of an avatar and/or an ambient lighting. The avatar can be presented on the display apparatus, for example. The ambient lighting can be modulated in order to provide intuitive feedback (e.g., green for good performance and red for poor performance of the at least one vehicle occupant).

The haptic feedback can be provided, for example, by way of steering wheel haptics, seat haptics, GUI haptics or combinations of these. The present disclosure is not limited thereto, however, and other haptic means can be used in order to output the feedback to the at least one vehicle occupant.

Preferably, the assistance system, in particular the output module, is designed to adjust at least one output parameter of the music output on the basis of the reaction of the at least one vehicle occupant in response to the music output. The at least one output parameter may be a volume, for example. By way of example, the volume can be increased for a poor performance of the vehicle occupant in order to subjectively improve the performance. The present disclosure is not limited to the volume, however, and, for example, a sound pattern of the music output can be adjusted.

In some embodiments, the at least one vehicle occupant is a single vehicle occupant, the reaction of whom in response to the music output is determined and output for feedback.

In other embodiments, the at least one vehicle occupant is two or more vehicle occupants. In this case, a respective reaction in response to the music output can be determined, and corresponding feedback can be output, for each of the two or more vehicle occupants.

The present disclosure is not limited thereto, however, and other lineups of vehicle occupants and/or driving assistance system (e.g., IPA) and/or external persons are conceivable for shared use of the assistance system, in particular the sing function or karaoke function.

In some embodiments, the assistance system can provide real-time feedback relating to the performance of multiple singers at the same time (e.g., a graphical user interface containing a bar chart, a star chart, a percentage rating, etc.), the identity of each singer being taken into consideration. This can be accomplished by virtue of the assistance system using facial recognition, for example, in order to recognize and identify the vehicle occupants. This can promote friendly competition and lead to an entertaining and agreeable experience.

In some embodiments, the assistance system can draw on earlier results or data relating to individual singers and can compare the general performance or the performance for specific songs sung in the past. This facilitates personal growth and a positive attitude for improvement compared with the competition.

Preferably, the assistance system comprises an intelligent personal assistant (IPA) designed for an interaction with the at least one vehicle occupant. In particular, the intelligent personal assistant may be designed to determine and output the feedback in response to the music output. Optionally, the IPA can appear as a participant in the karaoke.

The term “intelligent personal assistant” is understood to mean a software that can assist human beings in basic tasks and generally supplies information in natural language. IPAs can use online resources to answer the questions of a user, e.g., relating to the weather, relating to sports results, for providing route descriptions and for answering other questions. Within the context of the present disclosure, the functionality of the IPA can be extended in order to interact with the driver with regard to the performance during singing or to provide feedback and optionally to appear as a participant in the karaoke.

In particular, in some embodiments, the IPA can play an active role in the staging and moderation of the process (e.g., singing or karaoke) and/or may be a participant. As such, by way of example, the IPA can announce the music title, present the participants (vehicle occupants and optionally external users), provide a running commentary (e.g., in the form of voice, text, emojis, graphical feedback, etc.), announce the winners and praise (or criticize) the losers.

In some embodiments, the IPA can become a participant, and so the vehicle occupant can compete against the IPA, e.g., if travelling alone or if other persons in the vehicle do not wish to sing to a song (e.g., owing to preferences or because they do not know a song). Moreover, the IPA can allow the vehicle occupants to compete against the IPA together (e.g., in order to promote social cohesion among the vehicle occupants). Alternatively, the vehicle occupants may also compete among one another or as a team against one another.

Preferably, the assistance system is designed to receive a music selection from the at least one vehicle occupant. By way of example, the at least one vehicle occupant may select a song, or the assistance system may suggest and/or select a song (e.g., as a complement or contrast to the last song played).

The process, or the sing function (e.g., karaoke function), can be activated by a vehicle occupant, or the process, or the sing function (e.g., karaoke function), can be activated by the selection of the music. For example, in some embodiments, every song played can be started as karaoke in which the vehicle occupants compete against one another. Alternatively, the assistance system can use the IPA, for example, to recommend starting the process on the basis of the journey or one or more criteria, e.g., if the occupants have already been travelling for many hours, show signs of boredom or tiredness, etc.

In some embodiments, the assistance system may be designed to automatically make a music selection on the basis of at least one circumstance parameter. The at least one circumstance parameter may comprise or be, for example, a season (e.g., Christmas time, vacations, etc.), a date (e.g., Valentine's Day, birthday, Halloween, etc.), a time of day (morning or evening), an identity of the at least one vehicle occupant, etc.

Preferably, the assistance system is designed for real-time software updates. This allows the capacity of the assistance system to be updated, the training with new music content to be improved, and additional functions and/or GUI functions to be provided, during the period of use.

Preferably, use of the functionalities of the assistance system requires a subscription (e.g., monthly or annual payments). The functionalities of the assistance system may moreover be part of a larger subscription package or a vehicle option that is purchased with the vehicle or added at a later time during the possession cycle.

In some embodiments, a social media aspect and/or cloud aspect may be activatable in which the participants can play the game outside the vehicle or in another vehicle remotely with friends or the public (as in the case of social gaming). In particular, in some embodiments, the at least one vehicle occupant can play with at least one participant outside the vehicle.

According to another aspect of the present disclosure, a vehicle, in particular a motor vehicle, is specified. The vehicle comprises the assistance system according to the embodiments of the present disclosure.

The term vehicle covers passenger vehicles, trucks, buses, motor caravans, motorcycles, etc., used for conveying people, goods, etc. In particular, the term covers motor vehicles for conveying people.

Preferably, the vehicle is designed for automated driving. By way of example, the assistance system according to the invention may be activatable only if the vehicle is travelling in an automated driving mode. The present disclosure is not limited thereto, however, and the assistance system, or the sing function or karaoke function, may also be activatable or usable during manual driving.

The term “automated driving” may be understood within the context of the document to mean driving with automated longitudinal or lateral guidance or autonomous driving with automated longitudinal and lateral guidance. Automated driving may be for example driving for an extended period of time on the freeway or driving for a limited period of time when parking or maneuvering. The term “automated driving” covers automated driving with any desired level of automation. Illustrative levels of automation are assisted, partially automated, highly automated or fully automated driving. These levels of automation have been defined by the German Federal Highway Research Institute (BASt) (see BASt publication “Forschung kompakt”, issue November 2012).

In the case of assisted driving, the driver performs the longitudinal or lateral guidance on an ongoing basis, while the system undertakes the respective other function within certain boundaries. In the case of partially automated driving (TAF), the system undertakes the longitudinal and lateral guidance for a certain period of time and/or in specific situations, the driver needing to monitor the system on an ongoing basis as in the case of assisted driving. In the case of highly automated driving (HAF), the system undertakes the longitudinal and lateral guidance for a certain period of time without the driver needing to monitor the system on an ongoing basis; however, the driver must be capable of taking over vehicle guidance within a certain time. In the case of fully automated driving (VAF), the system can automatically cope with driving in all situations for a specific application; a driver is no longer needed for this application.

The aforementioned four levels of automation correspond to SAE levels 1 to 4 of SAE standard J3016 (SAE—Society of Automotive Engineering). Furthermore, SAE J3016 also has provision for SAE level 5 as the highest level of automation, which is not included in the definition from the BASt. SAE level 5 corresponds to driverless driving, in which the system can automatically cope with all situations throughout the journey in the same way as a human driver; a driver is generally no longer needed.

According to another independent aspect of the present disclosure, an assistance method is specified. The assistance method comprises outputting music in a vehicle interior; optically and/or acoustically detecting at least one vehicle occupant by way of an interior sensor system and providing corresponding detection data; determining a reaction, or behavior, of the at least one vehicle occupant in response to the music on the basis of the detection data; and controlling at least one vehicle function on the basis of the reaction, or behavior, of the at least one vehicle occupant in order to provide the at least one vehicle occupant with feedback relating to the reaction, or behavior, in response to the music output.

The assistance method can implement the aspects of the assistance system for a vehicle described in this document.

According to another independent aspect of the present disclosure, a software (SW) program is specified. The SW program can be designed to be executed on one or more processors and to thereby perform the assistance method described in this document.

According to another independent aspect of the present disclosure, a storage medium is specified. The storage medium may comprise an SW program designed to be executed on one or more processors and to thereby perform the assistance method described in this document.

According to another independent aspect of the present disclosure, a software containing program code for carrying out the assistance method is intended to be executed when the software runs on one or more software-controlled devices.

According to another independent aspect of the present disclosure, a system for providing vehicle-related data is specified. The system comprises one or more processors; and at least one memory that is connected to the one or more processors and contains instructions that can be executed by the one or more processors in order to perform the assistance method described in this document.

A processor, or a processor module, is a programmable arithmetic and logic unit, that is to say a machine or an electronic circuit that takes transferred commands as a basis for controlling other elements and advancing an algorithm (process).

Exemplary embodiments of the disclosure are depicted in the figures and are described in more detail below. In the figures:

### BRIEF DESCRIPTION OF THE DRAWINGS

FIG. 1 schematically shows an assistance system based on embodiments of the present disclosure,

FIG. 2 schematically shows a display apparatus containing feedback based on embodiments of the present disclosure, and

FIG. 3 shows a flowchart for an assistance method based on embodiments of the present disclosure.

### DETAILED DESCRIPTION OF THE DRAWINGS

Unless indicated otherwise, the same reference signs are used below for elements that are the same and have the same effect.

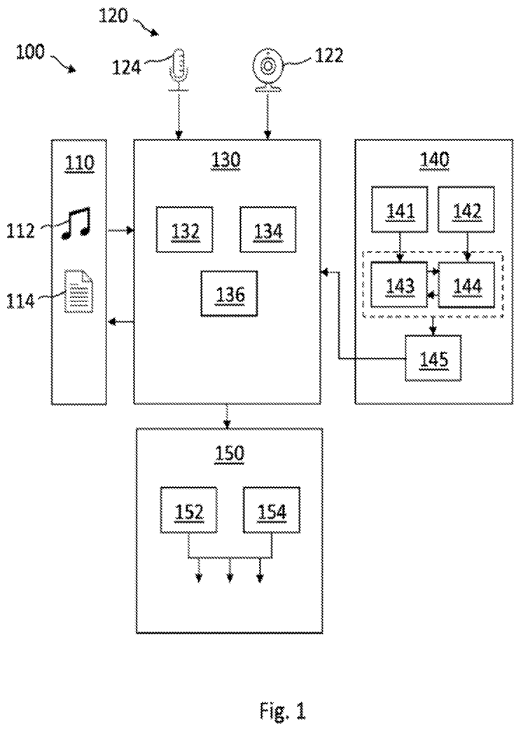

FIG. 1 schematically shows an assistance system 100 based on embodiments of the present disclosure.

A data source 110 provides musical acoustic data 112 (and optionally video data) for a music output, or music reproduction, in a vehicle interior. Typically, the music output, or the playback of the music, is provided by way of at least one loudspeaker in the interior of the vehicle (not shown).

In some embodiments, the data source 110 further provides lyrics data 114 for visually outputting a linguistic part of the music output. The visual output of the song texts is preferably provided by means of a display apparatus, for example, a graphical user interface (GUI) of the vehicle.

The provision of the musical acoustic data 112 and the lyrics data 114 can be implemented in at least two different ways: in a first implementation, the music and song texts are integrated in the assistance system 100 (internal data source 110). In a second implementation, the assistance system 100 operates in parallel with the music and the song texts provided by another system (external data source 110; e.g., an infotainment player, a third-party app running in the vehicle, CarPlay, Android Auto, a mobile terminal, etc.).

In the case of the second implementation, the music and/or the song texts are not integrated but rather are provided from outside as a stream. The assistance system 100 thus operates in parallel with an existing music player and uses the music player (e.g., Apple Music and Spotify supply song texts while the song is being played back). This approach of operating in parallel with the source of the music/texts can simplify the assistance system 100 and for example is not dependent on the provision of music and texts, the management of content or license issues.

The assistance system 100 further comprises an interior sensor system 120 designed to optically and/or acoustically detect at least one vehicle occupant and to provide corresponding detection data. The interior sensor system 120 may comprise at least one interior camera 122 and/or at least one interior microphone 124, for example. The at least one interior camera 122 can supply video data for analyzing reactions, or the behavior, of the vehicle occupant(s) (e.g. the singing performance). Similarly, the at least one interior microphone 124 can supply audio data for analyzing the singing performance (further).

The illustrative assistance system 100 further comprises an artificial intelligence (AI) module 130 designed to take the detection data from the interior sensor system as a basis for determining the reaction, or behavior, of the at least one vehicle occupant in response to the music output. In some embodiments, the AI module 130 can carry out facial and/or body recognition (block 132) in order to use the music and lyrics (block 134) to carry out an evaluation of the performance of the at least one vehicle occupant (block 136).

By way of example, the AI module 130 can analyze a degree of synchronization of the facial characteristics of one or more vehicle occupants (e.g., multiple vehicle occupants at the same time) with the text and/or music. The AI module 130 can implement lip reading, for example. In some embodiments, the degree of synchronization may comprise parameters such as timing and/or movement of the facial and/or body features (e.g., whether the mouth and lips move far enough apart for the given dynamics and volume of the music). It is conceivable for emotions to be recognized (e.g., by evaluating the camera data) at different times or periodically during the process, for example during singing, before and/or after singing, when the music is playing and no text needs to be sung at this time, etc.

Typically, the artificial intelligence module 130 comprises a trained algorithm. The algorithm can be trained by means of a machine learning (ML) module 140 on the basis of training data 141, for example. The larger the training dataset, the better a performance of the assistance system 100 becomes.

In the example of FIG. 1, the ML uses training data 141 to train the algorithm 143. Test data 142 are used for an evaluation 144, and so a trained algorithm or a model 145 is produced that is used to evaluate the performance of the at least one vehicle occupant.

In some embodiments, the algorithm can be trained on the basis of video data and/or audio data from single or multiple persons singing to different songs. Additionally or alternatively, the algorithm can be trained on the basis of musical acoustic data and/or lyrics data corresponding to the music output (i.e., the same songs). Additionally or alternatively, the algorithm can be trained on the basis of musical acoustic data and/or lyrics data similar to the music output (i.e., similar songs). Additionally or alternatively, the algorithm can be trained on the basis of historical data with regard to earlier music outputs and reactions of vehicle occupants in response thereto. In particular, the assistance system 100 may comprise an integrated training function, and so the assistance system 100 learns from the behavior of the actual users to a certain degree during the life of the assistance system 100.

Preferably, the algorithm can be dynamically updated. This allows the capacity of the algorithm to be updated, the training with new music content to be improved, and additional functions and/or GUI functions to be provided, during the period of use.

The AI module 130 is designed to take the detection data from the interior sensor system 120 as a basis, and to use the trained model 145, for determining a reaction, or behavior, of the at least one vehicle occupant in response to the music output. The reaction, or behavior, of the at least one vehicle occupant in response to the music output is taken as a basis for controlling at least one vehicle function 150 in order to provide the at least one vehicle occupant with feedback.

The at least one vehicle function may comprise an intelligent personal assistant (IPA) 152 and an audible and/or visual and/or haptic feedback module 154, for example. The audible feedback may comprise tones and/or a voice (e.g., of the IPA 152), for example. Additionally or alternatively, the visual feedback may comprise an avatar and/or can be provided by means of a display apparatus (e.g., GUI), for example. Additionally or alternatively, the haptic feedback can be provided by means of steering wheel haptics and/or an interior lighting, for example. Ultimately, a multisensory output can be provided.

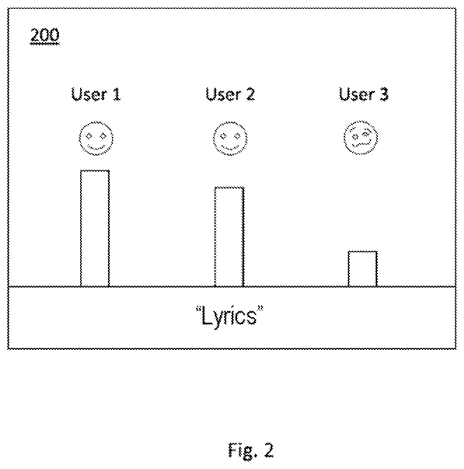

FIG. 2 schematically shows a display apparatus 200 containing feedback based on embodiments of the present disclosure.

In some embodiments, the assistance system can provide real-time feedback relating to the performance of multiple singers at the same time. In the example of FIG. 2, a graphical user interface containing a bar chart for three participants User 1, User 2 and User 3 and a song text (“Lyrics”) is depicted. The present disclosure is not restricted to this example, however, and other graphical elements (e.g., star charts, a percentage rating, emojis, etc.) and/or audible and/or haptic feedback elements can be used.

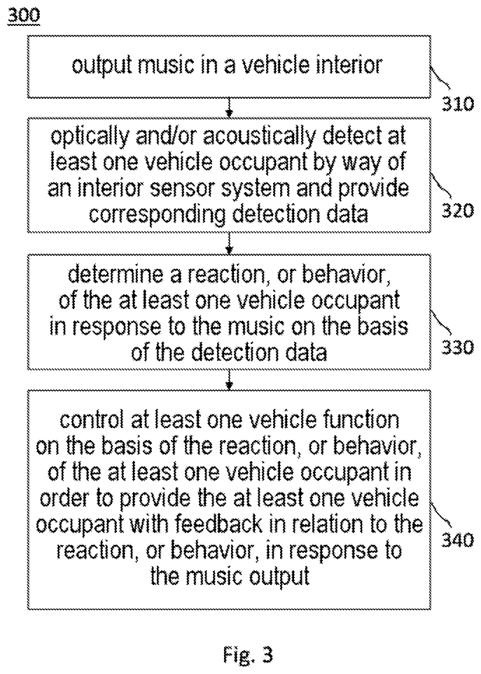

FIG. 3 shows a flowchart for an assistance method 300 based on embodiments of the present disclosure. The assistance method 300 can be implemented by an appropriate software that can be executed by one or more processors (e.g., a CPU).

The assistance method 300 comprises, in block 310, outputting music in a vehicle interior; in block 320, optically and/or acoustically detecting at least one vehicle occupant by way of an interior sensor system and providing corresponding detection data; in block 330, determining a reaction, or behavior, of the at least one vehicle occupant in response to the music on the basis of the detection data; and in block 340, controlling at least one vehicle function on the basis of the reaction, or behavior, of the at least one vehicle occupant in order to provide the at least one vehicle occupant with feedback in relation to the reaction, or behavior, in response to the music output.

According to the invention, a reaction, or behavior, of a vehicle occupant in response to played-back media content is detected and analyzed, e.g., as part of a sing function or karaoke function. The analysis is taken as a basis for providing the vehicle occupant with qualified feedback in relation thereto. By way of example, a performance of a single vehicle occupant or of multiple vehicle occupants can be assessed (at the same time or in succession) when singing along to music by assessing the behavior (expression, movement, dynamics, etc.) of various facial and/or body features and the synchronization with the song text and/or the music. This can be used to facilitate an intelligent interaction of the assistance system with the vehicle occupant with regard to played-back media content, e.g., as part of a sing function or karaoke function.

Although the invention has been illustrated and explained more thoroughly in detail by way of preferred exemplary embodiments, the invention is not restricted by the examples disclosed, and other variations can be derived therefrom by a person skilled in the art without departing from the scope of protection of the invention. It is therefore clear that there are a large number of variation possibilities. It is also clear that embodiments mentioned by way of illustration are actually only examples, which should in no way be regarded as a limitation of, for instance, the scope of protection, the application options or the configuration of the invention. Rather, the above description and the description of the figures enable a person skilled in the art to implement the illustrative embodiments in a concrete way, wherein the person skilled in the art, with knowledge of the concept of the invention disclosed, can make diverse changes for example with regard to the function or the arrangement of individual elements mentioned in an exemplary embodiment, without departing from the scope of protection defined by the claims and the legal counterparts thereof, such as, for instance, more extensive explanations in the description.

1.-15. (canceled)

16. An assistance system for a vehicle, comprising:
an output module designed for a music output based on musical acoustic data in a vehicle interior; and
an interior sensor system configured to optically and/or acoustically detect at least one vehicle occupant and to provide corresponding detection data;
wherein the assistance system is configured to:
take the detection data as a basis for determining a reaction of the at least one vehicle occupant in response to the music output; and
take the reaction of the at least one vehicle occupant in response to the music output as a basis for controlling at least one vehicle function in order to provide the at least one vehicle occupant with feedback in relation to the reaction in response to the music output.

17. The assistance system according to claim 16, wherein the output module is further configured to visually output a linguistic part of the music output based on lyrics data.

18. The assistance system according to claim 16, further comprising a storage module that has stored the musical acoustic data, wherein:
the storage module has further stored the lyrics data;
the storage module has permanently stored the musical acoustic data and/or the lyrics data; and/or
the storage module is integrated in the vehicle or an external unit connected to the vehicle, wherein the assistance system includes an interface module configured to receive the musical acoustic data and/or the lyrics data from the external unit.

19. The assistance system according to claim 16, wherein the assistance system is configured to receive the musical acoustic data and/or the lyrics data from an external unit and/or a streaming service.

20. The assistance system according to claim 16, wherein the reaction of the at least one vehicle occupant in response to the music output comprises at least one of the following elements:
a physical behavior of the at least one vehicle occupant, including a body expression, a body movement, a facial expression and/or a lip movement;
an acoustic performance of the at least one vehicle occupant with regard to the music output, including a volume, a rhythm and/or a register for singing along;
a synchronization between a singing along of the at least one vehicle occupant and the music output and/or the linguistic part of the music output; and
an emotional state of the at least one vehicle occupant.

21. The assistance system according to claim 16, further comprising an artificial intelligence module configured to take the detection data as a basis for determining the reaction of the at least one vehicle occupant in response to the music output.

22. The assistance system according to claim 21, wherein the artificial intelligence module comprises a trained algorithm, wherein the trained algorithm is trained based on at least one of the following aspects:
video data from single and multiple persons singing to different songs;
musical acoustic data and/or lyrics data corresponding to the music output;
musical acoustic data and/or lyrics data similar to the music output; and
historical data with regard to earlier music outputs and reactions of vehicle occupants in response thereto.

23. The assistance system according to claim 16, wherein the assistance system is configured to output the feedback to the at least one vehicle occupant audibly and/or visually and/or haptically.

24. The assistance system according to claim 16, wherein the assistance system is configured to adjust at least one output parameter of the music output based on the reaction of the at least one vehicle occupant in response to the music output, wherein the at least one output parameter comprises a volume.

25. The assistance system according to claim 16, wherein the at least one vehicle occupant is a single vehicle occupant, or
wherein the at least one vehicle occupant is two or more vehicle occupants, wherein a respective reaction in response to the music output is determined, and corresponding feedback is output, for each of the two or more vehicle occupants.

26. The assistance system according to claim 16, wherein the assistance system further comprises an intelligent personal assistant designed for an interaction with the at least one vehicle occupant, wherein the intelligent personal assistant is configured to determine the feedback in response to the music output.

27. The assistance system according to claim 16, wherein the assistance system is configured to receive a music selection from the at least one vehicle occupant and/or to automatically make a music selection based on at least one circumstance parameter.

28. A motor vehicle comprising an assistance system according to claim 16.

29. An assistance method, comprising:
outputting music in a vehicle interior;
optically and/or acoustically detecting at least one vehicle occupant by way of an interior sensor system and providing corresponding detection data;
determining a reaction of the at least one vehicle occupant in response to the music based on the detection data; and
controlling at least one vehicle function based on the reaction of the at least one vehicle occupant in order to provide the at least one vehicle occupant with feedback in relation to the reaction in response to the music output.

30. A non-transitory storage medium storing instructions to be executed on one or more processors to perform an assistance method according to claim 29.
